(19) United States
(12) Patent Application Publication
(10) Pub. No.: US 2026/0069660 A1
(43) Pub. Date: Mar. 12, 2026

(54) TNF ALPHA AND INTERLEUKIN-2 COMBINATION THERAPY FOR NON-MELANOMA SKIN CANCER

(71) Applicant: PHILOGEN S.P.A., Siena (IT)

(72) Inventors: Dario NERI, Siena (IT); Emanuele PUCA, Siena (IT); Giuliano ELIA, Siena (IT); Lisa NADAL, Siena (IT)

(21) Appl. No.: 19/108,072

(22) PCT Filed: Sep. 1, 2023
(86) PCT No.: PCT/EP2023/074056

§ 371 Date: Feb. 28, 2025

(30) Foreign Application Priority Data
Sep. 1, 2022 (EP) 22193432.6
Mar. 21, 2023 (EP) 23163294.4
Apr. 18, 2023 (EP) 23168600.7

(51) Int. Cl. A61K 38/20; A61K 38/19; A61K 47/68; A61P 35/00

(52) U.S. Cl. CPC A61K 38/2013; A61K 38/191; A61K 47/6849; A61P 35/00

(57) ABSTRACT

The application relates to the treatment of cancer, including non-melanoma skin cancer. The invention involves the use of a tumor necrosis factor alpha (TNFα) immunoconjugate and an interleukin 2 (IL2) immunoconjugate in a combination therapy for the treatment of non-melanoma skin cancer.

### CROSS-REFERENCE TO RELATED APPLICATION

This application is a § 371 national phase of International Application No. PCT/EP2023/074056, filed on Sep. 1, 2023, which claims priority from EP22193432.6 filed 1 Sep. 2022, from EP23163294.4 filed 21 Mar. 2023, and from EP23168600.7 filed 18 Apr. 2023, the contents and elements of which are herein incorporated by reference for all purposes.

### INCORPORATION BY REFERENCE OF SEQUENCE LISTING PROVIDED AS A SEQUENCE LISTINQ XML FILE

A Sequence Listing is provided herewith as a Sequence Listing XML, “008477200 xml”, created on Sep. 18, 2023, and having a size of 16,468 bytes. The contents of the Sequence Listing XML are incorporated herein by reference in their entirety.

### FIELD OF THE INVENTION

The present invention relates to a combination therapy for the treatment of non-melanoma skin cancer in which a tumor necrosis factor alpha (TNFα) immunoconjugate and an interleukin 2 (IL2) immunoconjugate are administered intratumorally.

### BACKGROUND

Many cytokines have shown potent antitumor activities in preclinical experiments and represent promising candidates for cancer therapy. Tumor necrosis factor alpha (TNFα) is a cytokine produced by many cell types, mainly activated monocytes and macrophages. It is expressed as a 26 kDa integral transmembrane precursor protein from which a mature protein of approximately 17 kDa is released by proteolytic cleavage. The soluble bioactive TNFα is a homotrimer that binds cell surface receptors. TNFα has been shown to induce necrosis of solid tumours. It exerts its effects mainly on the endothelium of the tumour-associated vasculature, with the effects including increased permeability, upregulation of tissue factor, fibrin deposition and thrombosis, and extensive destruction of endothelial cells.

Interleukin-2 (IL2), a four alpha helix bundle cytokine produced by T helper cells, plays an essential role in the activation phases of both adaptive and innate immune responses. Although this cytokine is not believed to have a direct cytotoxic effect on cancer cells, it has been reported to induce tumour regression by stimulating a cell-mediated immune response. In an attempt to increase the therapeutic index of certain cytokines, antibody-cytokine fusion proteins (also referred to as “immunocytokines” or “immunoconjugates”) have been developed. In these immunoconjugates, the antibody serves as a “vehicle” to drive selective accumulation of the cytokine at the site of disease, while the cytokine payload is responsible for the therapeutic activity (Pasche & Neri, 2012).

The current applicants described in WO01/062298 the immunoconjugates L19-huIL2 and L19-huTNFα, which comprise human interleukin 2 (huIL2) and human tumor necrosis factor alpha (huTNFα) respectively, each fused to the L19 antibody in single chain variable fragment (scFv) format (disclosed in WO99/058570). L19 specifically binds to the ED-B domain of fibronectin, one of the best-known markers of angiogenesis (U.S. Ser. No. 10/382,107; WO01/62298). ED-B is an extra domain of 91 amino acids which accumulates around neovascular structures in aggressive tumours and other tissues undergoing angiogenesis, such as the endometrium in the proliferative phase and some ocular structures in pathological conditions but is otherwise undetectable in healthy adult tissues.

The current applicants have described various uses of L19-huIL2 and of L19-huTNFα for the treatment of cancer in combination with a variety of other cancer therapeutics. For example, treatment of cancer with L19-huIL2 in combination with gemcitabine, antibodies against B-cell progenitors, CTLA-4 blockers, CAR-T cells, certain small-molecule drug conjugates, certain checkpoint inhibitors, and chemotherapy has been reported in WO2007/115837, WO2009/089858, WO2013/010749, WO2017/178562, WO2018/154517, WO2019/185792, and WO2021/234178 respectively.

In addition, L19-huIL2 and L19-huTNFα have been used in combination in cancer therapy. For example, L19-huIL2 and L19-huTNFα were shown to be able to cure neuroblastoma in a fully syngeneic mouse model of the disease, whereas the individual immunoconjugates used as single agents did not result in eradication of the disease (Balza et al., 2010). The combination of IL2 and TNFα payloads has also shown promising results in clinical trials. L19-huIL2 and L19-huTNFα were shown to potently synergize for the intralesional treatment of certain solid tumors in mice (Schwager et al., 2013). The corresponding fully human fusion proteins have been administered intralesionally to patients with Stage IIIC melanoma (Danielli et al., 2015), showing better results compared to the intralesional administration of interleukin-2 (Weide et al., 2011) or of L19-IL2 (Weide et al., 2014). In WO2013/045125 the current applicants described the administration of a single dose of L19-huIL2 and L19-huTNFα for the treatment of skin tumors.

Non-melanoma skin cancer refers (NMSC) to a group of cancers that slowly develop in the upper layers of the skin. The term non-melanoma distinguishes these common types of skin cancer from less common skin cancer known as melanoma, which can be more serious. Basal cell carcinoma (BCC) and cutaneous squamous cell carcinoma (cSCC) account for 99% of NMSC. Other NMSCs include Merkel cell carcinoma (MCC), Kaposi's sarcoma (KS), cutaneous T-Cell lymphoma (CTCL), Keratoacanthoma (KA), and malignant adnexal tumors of the skin (MATS). In particular, other NMSCs include Merkel cell carcinoma (MCC), Kaposi's sarcoma (KS), cutaneous T-Cell lymphoma (CTCL), and malignant adnexal tumors of the skin (MATS).

Complete surgical excision with histological control of margins remains the preferred treatment option for both BCC and cSCC due to their high five-year recurrence free survival rates. However, surgery is not always feasible depending on the anatomic location and is often not accepted by patients, as the procedure may be disfiguring, lead to deformation of the body and have adverse effects on the quality of life. Alternative treatment options for BCC are topical therapies (e.g., imiquimod or 5-fluoracil), photodynamic therapy or radiation therapy, which however do not produce a significant benefit in terms of recurrence free survival. Topical therapies with imiquimod or 5-fluoracil or photodynamic therapy are not recommended for cSCC, which further limits non-surgical treatment options.

While WO2013/045125 describes the intralesional administration of a single dose of L19-huIL2 and L19-huTNFα for the treatment of skin tumors, specifically malignant skin tumors, melanomas and carcinomas, it is silent about the treatment of NMSC, in particular treatment of NMSC in patients who are at risk of disfigurement following surgery, i.e. patients for whom surgery would produce an unacceptable cosmetic result.

Therefore, there remains a need in the art for effective treatments for NMSC, in particular treatments which do not result in the disfigurement often associated with surgical treatment.

The present invention has been devised in light of the above considerations.

### SUMMARY OF THE INVENTION

Reported here is the unexpected therapeutic effect resulting from intratumoral administration of a combination of L19-huTNFα and L19-huIL2 to a patient with a disfiguring BCC tumor on the nose. Surprisingly, the tumor showed a complete clinical and pathologic response to the treatment (Example 1).

The patient received four intratumoral injections of a mixture of 1.08 mg L19-huIL2 and 200 μg L19-huTNFα each in a volume of 1 ml. In the second injection, the dose of L19-huIL2 was accidentally doubled to 2.17 mg. This is not expected to have had a material impact on the outcome of the treatment and the same outcome is expected for patients treated with a dose of 1.08 mg L19-huIL2 throughout the treatment. In response to treatment, the tumor decreased in size, progressively converting into a necrotic scab and then a crust, which eventually fell off. Two months after the first injection, the tumor showed a complete clinical response and pathological response to treatment and the tumor was replaced by a fully epithelialized scar.

The complete clinical and pathological response of the tumor to the treatment was entirely unexpected and was observed as part of the first human trial of this combination therapy for the treatment of a non-melanoma skin cancer. As a result of the complete response of the tumor to the treatment, the patient was spared the amputation of the nose. The patient showed no recurrence of the cancer at a follow-up 6 months after the first injection.

The complete response of the BCC as a result of the intratumoral injection of a combination of L19-huIL2 and L19-huTNFα demonstrates the surprising efficacy of this combination for the treatment of non-melanoma skin cancers.

In a first aspect, the present invention thus relates to a composition comprising a combination of L19-TNFα and L19-IL2 for use in a method of treating a non-melanoma skin cancer in a patient by therapy.

The present invention also relates to a method of treating a non-melanoma skin cancer in a patient, the method comprising administering a therapeutically effective amount of a combination of L19-TNFα and L19-IL2 to the patient.

The present invention also relates to L19-TNFα for use in a method of treating a non-melanoma skin cancer in a patient by therapy, wherein the method comprises administering L19-TNFα in combination with L19-IL2.

The present invention further relates to L19-IL2 for use in a method of treating a non-melanoma skin cancer in a patient by therapy, wherein the method comprises administering L19-IL2 in combination with L19-TNFα.

Preferably, the method of treating a non-melanoma skin cancer in a patient comprises injecting the composition intratumorally.

The use of a combination of L19-TNFα and L19-IL2 for the manufacture of a medicament for the treatment of a non-melanoma skin cancer, is also disclosed.

Another aspect of the invention provides a therapeutic kit comprising a composition as described herein.

Preferably, the method comprises injecting a dose of the composition. For example, the dose of the composition may comprise 1 to 5 mg L19-IL2 and 100 to 500 μg L19-TNFα. Preferably, the dose of the composition comprises 1 to 2 mg L19-IL2 and 200 to 400 μg L19-TNFα. More preferably, the dose of the composition comprises 1 to 2.2 mg L19-IL2 and 200 to 400 μg L19-TNFα. In some preferred embodiments, the dose of the composition comprises 1.08 mg L19-IL2 and 200 μg L19-TNFα. In other preferred embodiments, the dose of the composition comprises 2.17 mg L19-IL2 and 400 μg L19-TNFα.

The dose of L19-IL2 can alternatively be stated in international units (IU). For example, the dose of the composition may comprise 5 million to 15 million IU L19-IL2 and 100 to 500 μg L19-TNFα. Preferably, the dose of the composition comprises 6 million to 13 million IU L19-IL2 and 200 to 400 μg L19-TNFα. More preferably, the dose of the composition may comprise 6.5 million to 13 million L19-IL2 and 200 to 400 μg L19-TNFα. 1.08 mg L19-IL2 equates to 6.5 million IU of L19-IL2. 2.17 mg L19-IL2 equates to 13 million IU L19-IL2. Thus, in some preferred embodiments, the dose of the composition comprises 6.5 million IU L19-IL2 and 200 μg L19-TNFα. In some alternative preferred embodiments, the dose of the composition comprises 13 million IU L19-IL2 and 400 μg L19-TNFα.

Preferably, the L19-TNFα is L19-huTNFα. More preferably, the L19-huTNFα comprises or consists of the amino acid sequence set forth in SEQ ID NO: 16.

Preferably, the L19-IL2 is L19-huIL2. More preferably, the L19-huIL2 comprises or consists of the amino acid sequence set forth in SEQ ID NO: 13.

Preferably the method of treating a non-melanoma skin cancer in a patient comprises injecting the composition intratumorally. In some preferred embodiments, a single dose of the composition is divided into multiple intratumoral injections, which are administered to the same tumor. This method of administration delivers the dose to different parts of the tumor.

Accordingly, a single dose of the composition may be administered in one or more intratumoral injections. Preferably, a single dose of the composition is administered in multiple intratumoral injections, which are administered to the same tumor, wherein each intratumoral injection is administered to a different segment of the tumor to distribute the dose across the tumor surface. The aim is for the dose to be distributed evenly over the surface of the tumor. The number of injections may thus be selected depending on the surface area of the tumor. For example, a single dose of the composition may be administered in 1 to 30, for example, 1 to 20, 1 to 15, 1 to 10, 1 to 9, 1 to 8, 1 to 7, 1 to 6, 1 to 5, 1 to 4, 1 to 3, or 1 to 2 intratumoral injections. In one example, a single dose may be administered in 1 to 6 intratumoral injections. Where a single dose of the composition is divided into two intratumoral injections, for example, each intratumoral injection may be administered to a different segment (preferably, a different half) of the tumor surface.

The non-melanoma skin cancer is preferably selected from the group consisting of: basal cell carcinoma (BCC), cutaneous squamous cell carcinoma (cSCC), Merkel cell carcinoma (MCC), Kaposi sarcoma (KS), cutaneous T-Cell lymphoma (CTCL), malignant adnexal tumors of the skin (MATS), and keratoacanthoma (KA). More preferably, the non-melanoma skin cancer is BCC, or cSCC. Most preferably, the non-melanoma skin cancer is BCC. In a preferred embodiment, the BCC is a stage IIA, IIB, IIIA, or IIIB BCC, defined according to the EADO classification system (Grob et al., 2021). Most preferably, the BCC is stage IIIB BCC, defined in according to the EADO classification system (Grob et al., 2021).

Preferably, the non-melanoma skin cancer is non-metastatic. In a preferred embodiment, the non-melanoma skin cancer is located on the patient's head. In a more preferred embodiment, the non-melanoma skin cancer is located on the patient's scalp, or face, or ears, in particular on the patient's face or ears, most preferably on the patient's face.

Thus, in a preferred embodiment, the non-melanoma skin cancer is non-metastatic BCC. In a preferred embodiment, the BCC is located on the patient's head. In a more preferred embodiment, the BCC is located on the patient's scalp, face or ears, in particular on the patient's face or ears, most preferably on the patient's face.

Similarly, in an alternative preferred embodiment, the non-melanoma skin cancer is cSCC, wherein the cSCC is non-metastatic. In a preferred embodiment, the cSCC, is located on the patient's head. In a more preferred embodiment, the cSCC is located on the patient's scalp, face or ears, in particular on the patient's face or ears, most preferably on the patient's face.

Preferably, the method of treating a non-melanoma skin cancer in a patient described herein reduces the size of the tumor.

Preferably, tumor size is measured using the volume of the tumor, or the area of the tumor on the surface of the patient's skin. Thus, the method can optionally include a step of sending the patient for observation of tumor size at a timepoint after the administration of the first dose of L19-TNFα and L19-IL2 to the patient, e.g. at least 10 days after, at least two weeks after, at least one month, at least two months, and/or at least 3 months, e.g. between one and three months, such as two months, after administration of the first dose of L19-TNFα and L19-IL2 to the patient. In one example, the method may optionally include a step of sending the patient for observation of tumor size at least 6 weeks, at least 9 weeks, at least 12 weeks, at least 20 weeks, at least 28 weeks, at least 36 weeks, at least 44 weeks, and/or at least 52 weeks after administration of the first dose of L19-TNFα and L19-IL2 to the patient. Preferably, a reduction in tumor size is observed at one or more of these time points. In a preferred embodiment, the method includes a step of sending the patient for observation of tumor size at least 6 weeks, e.g. between 6 and 9 weeks, such as 9 weeks after the administration of the first dose of L19-TNFα and L19-IL2. For example, the method may include a step of sending the patient for observation of tumor size at least 36 days, e.g. between 36 and 63 days, such as 63 days, after the administration of the first dose of L19-TNFα and L19-IL2 to the patient.

Preferably, the tumor shows a complete clinical response to the treatment. Most preferably, the tumor shows a complete pathological response to the treatment. A clinical complete response and/or pathological complete response is advantageous as it removes the need for surgical removal of the tumor, which may be disfiguring, e.g. due to the size and/or location of the tumor, and/or have an adverse effect on the quality of life of the patient. Surgery is particularly problematic in the context of treatment of tumors located on the head of the patient, in particular on the face of the patient, as surgical removal of tumors on the head and/or face may be particularly disfiguring. Thus, in one embodiment the method comprises injecting the composition at the site of the non-melanoma skin cancer tumor, wherein the tumor is located on the head of the patient, optionally wherein the tumor is located on the face of the patient.

In some embodiments, the non-melanoma skin cancer is not operable.

The method of treating a non-melanoma skin cancer can also include the subsequent step of taking a decision regarding further therapy, and optionally performing said further therapy, after viewing the results of the aforementioned tumor size observation. The skilled person is readily able to use techniques such as calliper measurement to perform this observation. Further therapy may comprise further administration of the composition of the present invention. Additionally, or alternatively, further therapy may comprise chemotherapy, radiotherapy and/or surgery. Preferably, further therapy comprises surgery. Thus, in some embodiments the treatment may be a neoadjuvant treatment. In some embodiments, the method comprises a step sending the patient for surgery, or performing surgery, at least 6 weeks e.g. between 6 and 12 weeks, after the administration of the first dose of L19-TNFα and L19-IL2 to the patient.

In some embodiments, the patient is one who has not received previous treatment with IL-2 or TNFα.

Preferably, the method comprises injecting more than one dose of the composition intratumorally. For example, the method may comprise injecting an initial dose of the composition and one or more further doses of the composition intratumorally. Preferably, the method comprises injecting an initial dose of the composition and three further doses of the composition. Preferably, the doses are administered once-weekly.

In some embodiments, the one or more further dose is the same (i.e. comprises the same amount of L19-IL2 and/or L19-TNFα) as the initial dose.

In some alternative embodiments, one or more of the further doses comprise more or less L19-IL2 and/or L19-TNFα than the initial dose. A reduction in the dose may be desirable if the patient experiences side-effects, such as inflammation, following administration of the initial dose. Alternatively, the initial dose may be reduced to see whether the patient tolerates the treatment, with subsequent doses being increased if the patient shows good tolerability. In one example, the one or more of the further doses may comprise double or half the amount of L19-IL2 and L19-TNFα relative to the amount of L19-IL2 and L19-TNFα in the initial dose.

In some embodiments, one or more of the further doses comprise more L19-IL2 and/or L19-TNFα than the initial dose. Preferably, one or more of the further doses comprise double the amount of L19-IL2 and/or L19-TNFα relative to the initial dose. For example, all further doses may comprise double the amount of L19-IL2 and/or L19-TNFα relative to the initial dose.

In some embodiments, one or more of the further doses comprise less L19-IL2 and/or L19-TNFα than the initial dose. Preferably, one or more of the further doses comprise half the L19-IL2 and/or L19-TNFα relative to the initial dose. For example, all further doses may comprise half the amount of L19-IL2 and/or L19-TNFα relative to the initial dose.

Where a patient has multiple non-melanoma skin cancer tumors, e.g. multiple BCC tumors, the total dose may be distributed among two or more of said tumors via intratumoral injection.

A patient, as referred to herein, is preferably a human patient.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows the response of a patient with a stage IIIB basal cell carcinoma (BCC) located on the nose at baseline and following administration of 4 intratumoral injections of L19-huTNFα and L19-huIL2, administered once weekly, as described in Example 1.

FIG. 2 shows a fully epithelialized scar on the nose of the patient described in Example 1, indicating no recurrence of the cancer at a follow-up four months after the first injection.

FIG. 3 shows histochemical staining of punch biopsies taken at the site of the original tumor from the patient described in Example 1. The punch biopsies were taken before treatment (left panel) and on day 63 after the first injection (right panel). The absence of any residual tumor cells in the biopsy taken on day 63, as determined by histological analysis, demonstrates that the patient achieved a complete pathological response.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Immunoconjugates

The composition of the present invention comprises L19-IL2 and L19-TNFα. L19-IL2 is an immunoconjugate comprising IL2, linked to the antibody molecule L19, which binds the ED-B domain of fibronectin. L19-TNFα is an immunoconjugate comprising TNFα, linked to the antibody molecule L19.

Preferably, L19 comprises the complementarity determining regions (CDRs) of the L19 antibody as indicated below.

VH CDR1
SEQ ID NO: 1
SFSMS
VH CDR 2
SEQ ID NO: 2
SISGSSGTTYYADSVKG
VH CDR 3
SEQ ID NO: 3
PFPYFDY
VL CDR 1
SEQ ID NO: 4
RASQSVSSSFLA
VL CDR 2
SEQ ID NO: 5
YASSRAT
VL CDR 3
SEQ ID NO: 6
QQTGRIPPT

Preferably, L19 comprises the L19 VH domain and/or the L19 VL domain. Amino acid sequences of the L19 VH and L19 VL domains are set out in SEQ ID NO: 7 and SEQ ID NO: 9, respectively.

Preferably the antibody molecule is a single chain Fv (scFv), a diabody, or other antibody fragment of low molecular weight and/or lacking an Fc region. These properties assist with targeting and tissue penetration of the immunoconjugate at the tumour site. Thus, L19-IL2 and L19-TNFα preferably comprise scFv-L19, which is an scFv comprising an L19 VH domain and an L19 VL domain, wherein the VH and VL are conjoined in a single polypeptide chain by a peptide linker sequence such as the 12-amino acid residue linker set out in SEQ ID NO: 8. Preferably, the scFv-L19 comprises or consists of the amino acid sequence set out in SEQ ID NO: 10.

An antibody molecule may comprise a VH domain having an amino acid sequence with at least 60%, 65%, 70%, 75%, 80%, 85%, 90%, 95% or 100% sequence identity with the amino acid sequence of the L19 VH domain as set out in SEQ ID NO: 7, and/or comprises a VL domain having an amino acid sequence with at least 60%, 65%, 70%, 75%, 80%, 85%, 90%, 95% or 100% sequence identity with the amino acid sequence of the L19 VL domain as set out in SEQ ID NO: 9. Preferably the antibody molecule is L19 in scFv format comprising an L19 VH domain as set out in SEQ ID NO: 7 and an L19 VL domain as set out in SEQ ID NO: 9. In a preferred embodiment, the antibody molecule is L19 scFv having the amino acid sequence set forth in SEQ ID NO: 10. An antibody molecule may comprise or consist of an amino acid sequence with at least 60%, 65%, 70%, 75%, 80%, 85%, 90%, 95% or 100% sequence identity with the amino acid sequence of the L19 scFv having the amino acid sequence as set out in SEQ ID NO: 10.

The composition of the invention may comprise modified forms of the L19 VH and/or VL domain, for example an antibody molecule may comprise the L19 VH or L19 VL domain in which 1, 2, 3, 4 or 5 amino acid substitutions have been made in a CDR and/or framework region, while retaining specific binding to fibronectin ED-B. In addition, an antibody molecule may comprise a linker in which 1, 2, 3, 4 or 5 amino acid substitutions have been made relative to the amino acid sequence as set out in SEQ ID NO: 8. Such amino acid substitutions are preferably conservative, e.g. substitution of one hydrophobic residue for another, one polar residue for another, arginine for lysine, glutamic for aspartic acid, or glutamine for asparagine.

A molecular linker such as a peptide may be used to join the cytokine to the antibody molecule, facilitating expression of all or part of the immunoconjugate as a fusion protein. An example of a suitable linker is set out in SEQ ID NO: 12 and SEQ ID NO: 15. An immunoconjugate may comprise a linker in which 1, 2, 3, 4 or 5 amino acid substitutions have been made relative to the amino acid sequences set out in SEQ ID NO: 12 or SEQ ID NO: 15. Where the antibody molecule is a single chain molecule, such as an scFv, the entire immunoconjugate polypeptide chain may conveniently be produced as a fusion protein. Without wishing to be bound by theory, for the TNFα immunoconjugate, the fusion proteins are thought to assembled into trimers, allowing TNFα to adopt its normal trimeric form.

L19-IL2 preferably comprises human IL2. The human IL2 preferably comprises or consists of the amino acid sequence set out in SEQ ID NO: 11. Antibody molecules are preferably human or humanised antibody molecules. The L19-huIL2 conjugate may comprise or consist of the amino acid sequence set out in SEQ ID NO: 13. An immunoconjugate molecule may comprise or consist of a polypeptide having an amino acid sequence with at least 60%, 65%, 70%, 75%, 80%, 85%, 90%, 95% or 100% sequence identity with the amino acid sequence of the L19-huIL2 as set out in SEQ ID NO: 13. An immunoconjugate may comprise or consist of a polypeptide in which 1, 2, 3, 4 or 5 amino acid substitutions have been made relative to the amino acid sequence set out in SEQ ID NO: 13.

L19-TNFα preferably comprises human TNFα. More preferably, L19-TNFα comprises the soluble form of the extracellular domain of human TNFα. The human TNFα preferably comprises or consists of the amino acid sequence set out in SEQ ID NO: 14. Antibody molecules are preferably human or humanised antibody molecules. The L19-huTNFα conjugate may comprise or consist of the amino acid sequence set out in SEQ ID NO: 16. An immunoconjugate molecule may comprise or consist of a polypeptide having an amino acid sequence with at least 60%, 65%, 70%, 75%, 80%, 85%, 90%, 95% or 100% sequence identity with the amino acid sequence of the L19-huTNFα as set out in SEQ ID NO: 16. An immunoconjugate may comprise or consist of a polypeptide in which 1, 2, 3, 4 or 5 amino acid substitutions have been made relative to the amino acid sequence set out in SEQ ID NO: 16.

### Indications

The cancer to be treated in accordance with the present invention is non-melanoma skin cancer (NMSC). NMSCs include Basal Cell Carcinoma (BCC), Cutaneous Squamous Cell Carcinoma (cSCC), Merkel Cell Carcinoma (MCC), Kaposi's Sarcoma (KS), Cutaneous T-Cell lymphoma (CTCL), Keratoacanthoma (KA), and Malignant adnexal tumors of the skin (MATS). In one example, the NMSC may be Basal Cell Carcinoma (BCC), Cutaneous Squamous Cell Carcinoma (cSCC), Merkel Cell Carcinoma (MCC), Kaposi's Sarcoma (KS), Cutaneous T-Cell lymphoma (CTCL), and Malignant adnexal tumors of the skin (MATS).

Preferably, the NMSC is BCC or cSCC. More preferably, the NMSC is BCC.

The BCC or cSCC may be localized (non-metastatic, node negative, single or multifocal) BCC or cSCC amenable to intratumoral injection. The BCC or cSCC may be high-risk BCC or cSCC.

BCC can be classified according to the stage of the disease, as set out in the EADO classification system version 1 described in Grob et al., 2021. In a preferred embodiment, the BCC is a stage IIA, IIB, IIIA, or IIIB BCC, most preferably a stage IIIB BCC. Stages IIA, IIB, IIIA, or IIIB of BCC are preferably defined according to the EADO classification system version 1 (Grob et al., 2021).

Preferably, the non-melanoma skin cancer is non-metastatic. In some embodiments, the non-melanoma skin cancer is located on the head of the patient. In some embodiments, the non-melanoma skin cancer is located on the face or ears, preferably the face, of the patient.

Thus, in some preferred embodiments, the non-melanoma skin cancer is BCC, wherein the BCC is non-metastatic. In some preferred embodiments, the non-melanoma skin cancer is BCC, wherein the BCC is located on the head of the patient. In some preferred embodiments, the non-melanoma skin cancer is BCC, wherein the BCC is located on the face or ears, preferably the face of the patient. In some preferred embodiments, the non-melanoma skin cancer is non-metastatic BCC located on the head of the patient. In some preferred embodiments, the non-melanoma skin cancer is non-metastatic BCC located on the face, scalp, or ears, preferably the face or ears, more preferably the face of the patient.

In some preferred embodiments, the non-melanoma skin cancer is cSCC, wherein the cSCC is non-metastatic. In some preferred embodiments, the non-melanoma skin cancer is cSCC, wherein the cSCC is located on the head of the patient. In some preferred embodiments, the non-melanoma skin cancer is cSCC, wherein the cSCC is located on the face or ears, preferably the face of the patient. In some preferred embodiments, the non-melanoma skin cancer is non-metastatic cSCC located on the head of the patient.

In some preferred embodiments, the non-melanoma skin cancer is non-metastatic cSCC located on the face, scalp, or ears of the patient, preferably the face or ears, more preferably the face of the patient.

In some preferred embodiments, the treatment reduces the size of the non-melanoma skin cancer tumor. For example, the method may reduce the size of the non-melanoma skin cancer tumor by at least 5% 10%, 15%, 20%, 25%, 30%, 35%, 40%, 45%, 50%, 55%, 60%, 65%, 70%, 75%, 80%, 85%, 90%, 95% or 100%. Preferably, the treatment reduces the size of the non-melanoma skin cancer tumor by at least 50%. More preferably the method reduces the size of the non-melanoma skin cancer tumor by at least 90%. Tumor size in this context may refer to tumor volume. Alternatively, tumor size may refer to the tumor area visible on the surface of the patient's skin.

More preferably, the non-melanoma skin cancer tumor shows a clinical complete response to the treatment. Most preferably, the non-melanoma skin cancer tumor shows a pathological complete response to the treatment. The determination of a clinical or pathological complete response is within the capabilities of the skilled person. A clinical complete response (cCR) may refer to the absence of detectable tumor, as determined by imaging, such as by computed tomography (CT). A pathological complete response (pCR) may refer to the absence of detectable cancer cells in tissue samples obtained from the site of the original tumor. The presence of cancer cells in tissue samples may be determined using histological analysis.

### Administration

The dose is an amount of TNFα and IL2 cytokine in the form of immunoconjugates as described above, effective to treat the tumour in the combination therapy according to the invention.

In some embodiments, the dose of the composition comprises 0.5 to 5 mg L19-IL2 and 100 to 500 μg L19-TNFα. In some embodiments, the dose of the composition comprises 0.5 to 3 mg L19-IL2 and 150 to 450 μg L19-TNFα. In some embodiments, the dose of the composition comprises 1 to 2.2 mg L19-IL2 and 200 to 400 μg L19-TNFα. For example, the dose of the composition may comprise 1, 1.08, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.17, or 2.2 mg L19-IL2. The dose of the composition may further comprise 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, or 400 μg L19-TNFα. Preferably, the dose of the composition comprises 1.08 mg L19-IL2 and 200 μg L19-TNFα. In an alternative preferred embodiment, the dose of the composition comprises 2.17 mg L19-IL2 and 400 μg L19-TNFα.

The dose of L19-IL2 can alternatively be stated in international units (IU). For example, the dose of the composition may comprise 5 million to 15 million IU L19-IL2 and 100 to 500 μg L19-TNFα. For example, the dose of the composition may comprise 5 million, 5.5 million, 6 million, 6.5 million, 7 million, 7.5 million, 8 million, 8.5 million, 9 million, 9.5 million, 10 million, 10.5 million, 11 million, 11.5 million, 12 million, 12.5 million, 13 million, 13.5 million, 14 million, 14.5 million, or 15 million IU L19-IL2. Preferably, the dose of the composition comprises 6 to 13 million IU L19-IL2 and 200 to 400 μg L19-TNFα. More preferably, the dose of the composition comprises 6.5 million to 13 million L19-IL2 and 200 to 400 μg L19-TNFα. 1.08 mg L19-IL2 equates to 6.5 million IU L19-IL2. 2.17 mg L19-IL2 equates to 13 million IU L19-IL2. Thus, in some preferred embodiments, the dose of the composition comprises 6.5 million IU L19-IL2 and 200 μg L19-TNFα. In some alternative preferred embodiments, the dose of the composition comprises 13 million IU L19-IL2 and 400 μg L19-TNFα.

In some embodiments, the non-melanoma skin cancer is selected from the list consisting of Merkel cell carcinoma (MCC), Kaposi sarcoma (KS), cutaneous T-Cell lymphoma (CTCL), malignant adnexal tumors of the skin (MATS), and keratoacanthoma (KA) and the dose of the composition comprises 2.17 mg L19-IL2 and 400 μg L19-TNFα.

L19-IL2 may be optionally formulated at a dose of 2.17 mg in a total volume of 1.0 mL, while L19-TNFα may be optionally formulated at a dose of 400 μg in a total volume of 1.0 mL. For the preparation of the solution for intratumoral injection, the full volume of L19-IL2 may be transferred to the vial containing L19-TNFα (or vice versa), producing a solution of 2.17 mg L19-IL2+400 μg L19-TNFα in a total volume of 2.0 mL.

After gently mixing the solution, a volume of 1.0 mL containing 1.08 mg L19-IL2+200 μg L19-TNFα may be used for the intratumoral injection using a single syringe with a 30-gauge needle and the residual volume discarded. Alternatively, volume of 2.0 mL containing 2.17 mg L19-IL2+400 μg L19-TNFα may be used for the intratumoral injection using a single syringe with a 30-gauge needle and the residual volume discarded.

Preferably the method of treating a non-melanoma skin cancer in a patient comprises injecting the composition at the site of a non-melanoma skin cancer tumor, preferably by intratumoral injection.

Intratumoral injection is injection into the tumor. Peritumoral injection, e.g. local intradermal injection, is another suitable method for administering the composition locally to a tumor site.

In some preferred embodiments, a single dose of the composition is divided and injected intratumorally at multiple sites into a single non-melanoma skin cancer tumor. In this case the total volume of the dose is distributed across the surface area of the tumor or the tumor mass, e.g. the tumor mass.

In other preferred embodiments, the method comprises injecting the dose of the composition into more than one non-melanoma skin cancer tumor of the patient, wherein the dose is divided between the tumors. The total volume of the dose may be distributed between the tumors according to the following scheme:

| Number of tumors | Volume injected per tumor |
|---|---|
| 1 | 100% of L19-IL2 + L19-TNFα volume |
| 2 | 50% of L19-IL2 + L19-TNFα volume |
| 3 | 33% of L19-IL2 + L19-TNFα volume |

Thus, in some embodiments, the method comprises injecting the dose into two non-melanoma skin cancer tumors, wherein 50% of the dose is injected into each tumor. In some embodiments, the method comprises injecting the dose into three non-melanoma skin cancer tumors, wherein 33% of the dose is injected into each tumor. In this case, the total volume of the dose may be divided equally among all tumors irrespectively of their size.

In patients with four or more injectable non-melanoma skin cancer tumors, the tumors may be prioritized according to their size. Thus, in some embodiments wherein a patient has more than three non-melanoma skin cancer tumors, the method comprises injecting the dose into the three largest tumors, wherein 33% of the dose is injected into each of the three largest tumors. In this case, other smaller tumors may remain uninjected. In some embodiments, the three largest tumors may receive an equal dose in each tumor, irrespective of their size, so that the dosage per tumor is constant. In other embodiments, the largest tumor may be injected first, and all other tumors may then be prioritized for injection in decreasing tumor size, until the total volume has been injected. The size of a non-melanoma skin cancer tumor preferably refers to the longest (i.e. maximum) diameter of the tumor on the surface of the patient's skin (referred to herein as “longest diameter”).

Newly occurring non-melanoma skin cancer tumors within the 4-week treatment period may also be treated as described above. For the new tumors, the treatment period may not be extended beyond the four week-treatment period starting with the first intratumoral injection of L19-IL2 and L19-TNFα.

In some embodiments, the method comprises injecting a dose of the composition comprising a maximum amount of 2.2 mg L19-huIL2 and a maximum amount of 400 μg L19-TNFα. In some preferred embodiments, the method comprises injecting a dose of the composition comprising a maximum amount of 1.08 mg L19-IL2 and a maximum amount of 200 μg L19-TNFα. In some other preferred embodiments, the method comprises injecting a dose of the composition comprising a maximum amount of 2.17 mg L19-IL2 and a maximum amount of 400 μg L19-TNFα. The dose of the composition to be injected into a non-melanoma skin cancer tumor of a patient may be adjusted according to the size (e.g longest diameter) and/or the location of the tumor on the patient's body. For example, the dose of the composition to be injected into a non-melanoma skin cancer tumor of a patient may be reduced relative to one of the maximum amounts of L19-IL2 and L19-TNFα set out above.

In some patients, inflammation has been observed around the injection site following administration of the composition. Surprisingly, the present inventors have observed that an adjustment (reduction) of the dose of the composition to be injected into a tumor according to the size (e.g. longest diameter) and/or location of the tumor on the patient's body can reduce said inflammation around the injection site while still achieving the treatment effect, such as a complete clinical and/or pathological response, but preferably a pathological complete response, of the tumor.

For example, 50% of the maximum amount of L19-IL2 and L19-TNFα, which in this case was 1.08 mg L19-IL2 and 200 μg L19-TNFα, was injected intratumorally into a cSCC tumor located on an eyelid of a patient. Surprisingly, despite receiving a half-maximal amount of the composition (i.e. 0.54 mg L19-IL2 and 100 μg L19-TNFα), the tumor showed a complete pathologic response to the treatment.

The present inventors have also shown that the dose of the composition to be injected intratumorally can be reduced according to the size (e.g. longest diameter) of a BCC tumor of a patient while still achieving a pathologic complete response.

In instances where treatment of the tumors is prioritized according to their size, the volume of the dose may be administered according to the following scheme:

| Tumor - longest diameter (cm) | Volume to be injected |
|---|---|
| diameter > 3.0 | 100% of L19-IL2 + L19-TNFα volume |
| 2.0 < diameter ≤ 3.0 | 75% of L19-IL2 + L19-TNFα volume |
| 1.0 < diameter ≤ 2.0 | 50% of L19-IL2 + L19-TNFα volume |
| 0 < diameter ≤ 1.0 | 25% of L19-IL2 + L19-TNFα volume |

Thus, in some embodiments, the method comprises injecting the dose into a non-melanoma skin cancer tumor with a longest diameter of more than 3 cm, wherein 100% of the dose is injected into the tumor. In some embodiments, the method comprises injecting the dose into a non-melanoma skin cancer tumor with a longest diameter of more than 2 cm and less than or equal to 3 cm, wherein 75% of the dose is injected into the tumor. In some embodiments, the method comprises injecting the dose into a non-melanoma skin cancer tumor with a longest diameter of more than 1 cm and less than or equal to 2 cm, wherein 50% of the dose is injected into the tumor. In some embodiments, the method comprises injecting the dose into a non-melanoma skin cancer tumor with a longest diameter of more than 0 cm, e.g. at least 0.1 cm, such as between 0.1 cm and 1 cm, wherein 25% of the dose is injected into the tumor.

In some embodiments, the method comprises injecting the dose into a non-melanoma skin cancer tumor located on the nose or in a periorificial region of the head of a patient. The periorificial regions of the head of a patient can be determined by a clinician, e.g. in accordance with Pons et al, 2022. In patients with a non-melanoma skin cancer tumor located on the nose or in a periorificial region of the head of a patient, the volume of the dose of the composition to be injected into the tumor may be adjusted according to the size (e.g. longest diameter) of the tumor in accordance with the following scheme:

| Tumor located on the | |
|---|---|
| nose or in a periorificial | |
| region of the head - | |
| longest diameter (cm) | Volume to be injected |
| 1.0 < diameter ≤ 2.0 | 25% of L19-IL2 + L19-TNFα volume |
| 2.0 < diameter ≤ 3.0 | 37.5% of L19-IL2 + L19-TNFα volume |
| 3.0 < diameter ≤ 4.0 | 50% of L19-IL2 + L19-TNFα volume |
| 4.0 < diameter ≤ 5.0 | 62.5% of L19-IL2 + L19-TNFα volume |
| >5.0 diameter | 75% of L19-IL2 + L19-TNFα volume |

Thus, in some embodiments, the method comprises injecting a non-melanoma skin cancer tumor located on the nose or in a periorificial region of the head of the patient, the method comprising injecting a dose of the composition comprising:
(a) 25% of the maximum amount of L19-IL2 and L19-TNFα as set out above, where the tumor has a surface area with a longest diameter of >1 cm and ≤2 cm;
(b) 37.5% of the maximum amount of L19-IL2 and L19-TNFα as set out above, where the tumor has a surface area with a longest diameter of >2 cm and ≤3 cm;
(c) 50% of the maximum amount of L19-IL2 and L19-TNFα as set out above, where the tumor has a surface area with a longest diameter of >3 cm and ≤4 cm;
(d) 62.5% of the maximum amount of L19-IL2 and L19-TNFα as set out above, where the tumor has a surface area with a longest diameter of >4 cm and ≤5 cm; or
(e) 75% of the maximum amount of L19-IL2 and L19-TNFα as set out above, where the tumor has a surface area with a longest diameter of >5 cm.

Alternatively, in patients with a non-melanoma skin cancer tumor located on the nose or in a periorificial region of the head of the patient, the volume of the dose of the composition to be injected into the tumor may be adjusted according to the size (e.g. longest diameter) of the tumor in accordance with the following alternative scheme:

| Tumor located on the | |
|---|---|
| nose or in a periorificial | |
| region of the head - | |
| longest diameter (cm) | Volume to be injected |
| 1.0 < diameter ≤ 2.0 | 25% of L19-IL2 + L19-TNFα volume |
| 2.0 < diameter ≤ 3.0 | 37.5% of L19-IL2 + L19-TNFα volume |
| 3.0 < diameter ≤ 5.0 | 50% of L19-IL2 + L19-TNFα volume |
| >5.0 diameter | 62.5% of L19-IL2 + L19-TNFα volume |

Thus, in some alternative embodiments, the method comprises injecting a non-melanoma skin cancer tumor located on the nose or in a periorificial region of the head of the patient, the method comprising injecting a dose of the composition comprising:
(a) 25% of the maximum amount of L19-IL2 and L19-TNFα as set out above, where the tumor has a surface area with a longest diameter of >1 cm and ≤2 cm;
(b) 37.5% of the maximum amount of L19-IL2 and L19-TNFα as set out above, where the tumor has a surface area with a longest diameter of >2 cm and ≤3 cm;
(c) 50% of the maximum amount of L19-IL2 and L19-TNFα as set out above, where the tumor has a surface area with a longest diameter of >3 cm and ≤5 cm; or
(d) 62.5% of the maximum amount of L19-IL2 and L19-TNFα as set out above, where the tumor has a surface area with a longest diameter of >5 cm.

In some embodiments, the method comprises injecting the dose into a non-melanoma skin cancer tumor located on a part of the body other than on the nose or in a periorificial region of the head of the patient. For example, the method may comprise injecting the dose into a non-melanoma skin cancer tumor located on a region of the head of the patient other than on the nose or in a periorificial region, such as on the forehead, cheek, chin, or scalp of the patient. In patients with a non-melanoma skin cancer tumor located on a part of the body other than on the nose or in a periorifical region of the head of the patient, the volume of the dose of the composition to be injected into the tumor may be adjusted according to the size (e.g. longest diameter) of the tumor according to the following scheme:

| Tumor located on the | |
|---|---|
| forehead, cheek, chin, | |
| scalp or other part of | |
| the body (other than on the | |
| nose or in a periorificial | |
| region of the head) - | |
| longest diameter (cm) | Volume to be injected |
| 1.0 < diameter ≤ 2.0 | 37.5% of L19-IL2 + L19-TNFα volume |
| 2.0 < diameter ≤ 3.0 | 50% of L19-IL2 + L19-TNFα volume |
| 3.0 < diameter ≤ 4.0 | 62.5% of L19-IL2 + L19-TNFα volume |
| 4.0 < diameter ≤ 5.0 | 75% of L19-IL2 + L19-TNFα volume |
| >5.0 diameter | 100% of L19-IL2 + L19-TNFα volume |

Thus, in some embodiments, the method comprises injecting a non-melanoma skin cancer tumor located on a part of the body of the patient, such as the head, other than on the nose or in a periorificial region of the head of the patient, the method comprising injecting a dose of the composition comprising:
(a) 37.5% of the maximum amount of L19-IL2 and L19-TNFα as set out above, where the tumor has a surface area with a longest diameter of >1 cm and ≤2 cm;
(b) 50% of the maximum amount of L19-IL2 and L19-TNFα as set out above, where the tumor has a surface area with a longest diameter of >2 cm and ≤3 cm;
(c) 62.5% of the maximum amount of L19-IL2 and L19-TNFα as set out above, where the tumor has a surface area with a longest diameter of >3 cm and ≤4 cm;
(d) 75% of the maximum amount of L19-IL2 and L19-TNFα as set out above, where the tumor has a surface area with a longest diameter of >4 cm and ≤5 cm; or
(e) 100% of the maximum amount of L19-IL2 and L19-TNFα as set out above, where the tumor has a surface area with a longest diameter of >5 cm.

Alternatively, in patients with a non-melanoma skin cancer tumor located on a part of the body, such as the head, other than the nose or in a periorificial region of the head of the patient, the volume of the dose of the composition to be injected into the tumor may be adjusted according to the size (e.g. longest diameter) of the tumor according to the following alternative scheme:

| Tumor located on the | |
|---|---|
| forehead, cheek, chin, | |
| scalp or other part of | |
| the body (other than on the | |
| nose or in a periorificial | |
| region of the head) - | |
| longest diameter (cm) | Volume to be injected |
| 1.0 < diameter ≤ 2.0 | 37.5% of L19-IL2 + L19-TNFα volume |
| 2.0 < diameter ≤ 3.0 | 50% of L19-IL2 + L19-TNFα volume |
| 3.0 < diameter ≤ 5.0 | 75% of L19-IL2 + L19-TNFα volume |
| >5.0 diameter | 100% of L19-IL2 + L19-TNFα volume |

Thus, in some alternative embodiments, the method comprises injecting a non-melanoma skin cancer tumor located on a part of the body, such as the head, of the patient other than on the nose or in a periorificial region of the head of the patient, the method comprising injecting a dose of the composition comprising:
(a) 37.5% of the maximum amount of L19-IL2 and L19-TNFα as set out above, where the tumor has a surface area with a longest diameter of >1 cm and ≤2 cm;
(b) 50% of the maximum amount of L19-IL2 and L19-TNFα as set out above, where the tumor has a surface area with a longest diameter of >2 cm and ≤3 cm;
(c) 75% of the maximum amount of L19-IL2 and L19-TNFα as set out above, where the tumor has a surface area with a longest diameter of >3 cm and ≤5 cm; or
(d) 100% of the maximum amount of L19-IL2 and L19-TNFα as set out above, where the tumor has a surface area with a longest diameter of >5 cm.

In patients with multiple non-melanoma skin cancer tumors, the tumors may be prioritized according to their size. Thus, in some embodiments wherein a patient has multiple non-melanoma skin cancer tumors, the method comprises injecting each tumor, in order of decreasing tumor size as measured by the longest diameter of the tumor surface area, with a dose of the composition according to one of the dosage schemes above, until either: (i) the maximum amount of L19-IL2 and L19-TNFα as set out above has been injected; or (ii) all non-melanoma skin cancer tumors of the patient have been injected. In case (i), other smaller non-melanoma skin cancer tumors of the patient may remain uninjected. In case (ii), the residual volume of the dose may be discarded.

In patients with a single non-melanoma skin cancer tumor, wherein the volume to be injected into the patient's tumor according to one of the above schemes is <100% of the total volume of the dose, the residual volume of the dose may be discarded.

Thus, in patients with a single non-melanoma skin cancer tumor or multiple non-melanoma skin cancer tumors, wherein the total volume injected intratumorally (i.e. the sum of the volume injected into to each of the patient's tumor(s)) according to one of the above schemes is <100% of the total volume of the dose, the residual dose is discarded. Thus, in some embodiments, the method comprises injecting 25%, 37.5%, 50%, 62.5%, 75%, 87.5%, or 100% of the maximum amount of L19-IL2 and L19-TNFα as set out above intratumorally.

Thus, in some embodiments, the method comprises injecting a dose of the composition comprising 0.25 mg to 2.2 mg L19-IL2 and 50 μg to 400 μg L19-TNFα.

In some preferred embodiments, the method comprises injecting a dose of the composition comprising 0.27 mg to 1.08 mg L19-IL2 and 50 μg to 200 μg L19-TNFα. For example, the dose may comprise: 0.27 mg L19-IL2 and 50 μg L19-TNFα; 0.41 mg L19-IL2 and 75 μg L19-TNFα; 0.54 mg L19-IL2 and 100 μg L19-TNFα; 0.68 mg L19-IL2 and 125 μg L19-TNFα; 0.81 mg L19-IL2 and 150 μg L19-TNFα; 0.95 mg L19-IL2 and 175 μg L19-TNFα; or 1.08 mg L19-IL2 and 200 μg L19-TNFα. In other words, the dose may comprise 25%, 37.5%, 50%, 62.5%, 75%, 87.5%, or 100% of a maximum amount of L19-IL2 and L19-TNFα of 1.08 mg L19-IL2 and 200 μg L19-TNFα.

In other preferred embodiments, the method comprises injecting a dose of the composition comprising 0.54 mg to 2.17 mg L19-IL2 and 100 μg to 400 μg L19-TNFα. For example, the dose may comprise: 0.54 mg L19-IL2 and 100 μg L19-TNFα; 0.81 mg L19-IL2 and 150 μg L19-TNFα; 1.08 mg L19-IL2 and 200 μg L19-TNFα; 1.36 mg L19-IL2 and 250 μg L19-TNFα; 1.63 mg L19-IL2 and 300 μg L19-TNFα; 1.90 mg L19-IL2 and 350 μg L19-TNFα; or 2.17 mg L19-IL2 and 400 μg L19-TNFα. In other words, the dose may comprise 25%, 37.5%, 50%, 62.5%, 75%, 87.5%, or 100% of a maximum amount of L19-IL2 and L19-TNFα of 2.17 mg L19-IL2 and 400 μg L19-TNFα.

In some embodiments, the method comprises injecting an initial dose of the composition and one or more further doses of the composition intratumorally. Preferably, the doses are administered once weekly. For example, the dose may be administered once weekly for one to four weeks, e.g., for one, two, three or four consecutive weeks. In a preferred embodiment, the dose is administered once weekly for four weeks. In one embodiment, the dose is not administered for more than four weeks. The dose administered may be determined in accordance with one of the above schemes at each treatment point.

In some embodiments, the method comprises injecting an initial dose of the composition and one or more further doses of the composition intratumorally, and wherein one or more of the further doses comprises the same amount of L19-IL2 and/or L19-TNFα as the initial dose. For example, in the context of the schemes above, where the initial dose is determined according to the location and size (e.g. longest diameter) of the tumor, one or more of the further doses may comprise the same amount of L19-IL2 and/or L19-TNFα as the initial dose.

Alternatively, in some embodiments, the method comprises injecting an initial dose of the composition and one or more further doses of the composition intratumorally, and wherein one or more of the further doses comprise more or less L19-IL2 and/or L19-TNFα than the initial dose. For example, in the context of the schemes above, where the initial dose is determined according to the location and size (e.g. longest diameter) of the tumor, one or more of the further doses may comprise more or less L19-IL2 and/or L19-TNFα than the initial dose, wherein the one or more further doses administered do not exceed the maximum amount of L19-IL2 and L19-TNFα set out above. For example, one or more of the further doses may comprise double or half the amount of L19-IL2 and/or L19-TNFα relative to the initial dose, wherein the one or more further doses administered do not exceed the maximum amount of L19-IL2 and L19-TNFα set out above.

Dosage escalation for the subsequent administrations may be desired in the case of good tolerability of the initial dose. In some embodiments, the method comprises injecting an initial dose of the composition and one or more further doses of the composition intratumorally, and wherein one or more of the further doses comprise more L19-IL2 and/or L19-TNFα than the initial dose, wherein the one or more further doses administered do not exceed the maximum amount of L19-IL2 and L19-TNFα set out above. Preferably, one or more of the further doses comprise double the amount of L19-IL2 and/or L19-TNFα relative to the initial dose, wherein the one or more further doses administered do not exceed the maximum amount of L19-IL2 and L19-TNFα set out above.

Dosage reduction for the subsequent administrations may be required in the case of an adverse reaction (such as inflammation) at the injection site. In some embodiments, the method comprises injecting an initial dose of the composition and one or more further doses of the composition intratumorally, and wherein one or more of the further doses comprise less L19-IL2 and/or L19-TNFα than the initial dose. Preferably, the method comprises injecting an initial dose of the composition and one or more further doses of the composition intratumorally, and wherein one or more of the further doses comprise half the amount of L19-IL2 and/or L19-TNFα relative to the initial dose.

Some embodiments of this invention involve the use of the combination of IL2 and TNFα immunoconjugates formulated as a pharmaceutical composition. Pharmaceutical compositions may include a pharmaceutically acceptable “excipient” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. The excipients may include solvents, solubility enhancers, suspending agents, buffering agents, isotonicity agents, antioxidants or antimicrobial preservatives. Thus, the invention also provides a pharmaceutical composition comprising a combination of L19-huTNFα and L19-huIL2 for use in a method of treating a non-melanoma skin cancer in a patient, wherein the pharmaceutical composition comprises a pharmaceutically acceptable excipient.

### Kits

Another aspect of the invention provides a therapeutic kit for use in the treatment of a disease or disorder as described herein, comprising a composition as described herein. The components of a kit are preferably sterile and in sealed vials or other containers. In some embodiments, the therapeutic kit comprises a composition as described herein, wherein L19-TNFα and L19-IL2 are contained separately, e.g. in separate sealed vials. In some alternative embodiments, the therapeutic kit comprises a composition as described herein, wherein L19-TNFα and L19-IL2 are contained together, e.g. in the same sealed vial.

A kit may further comprise instructions for use of the components in a method described herein. The components of the kit may be comprised or packaged in a container, for example a bag, box, jar, tin or blister pack.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1—Intratumoral Combination L19-huIL2 and L19-huTNFα in a BCC Patient

### 1.1 Experimental Procedure

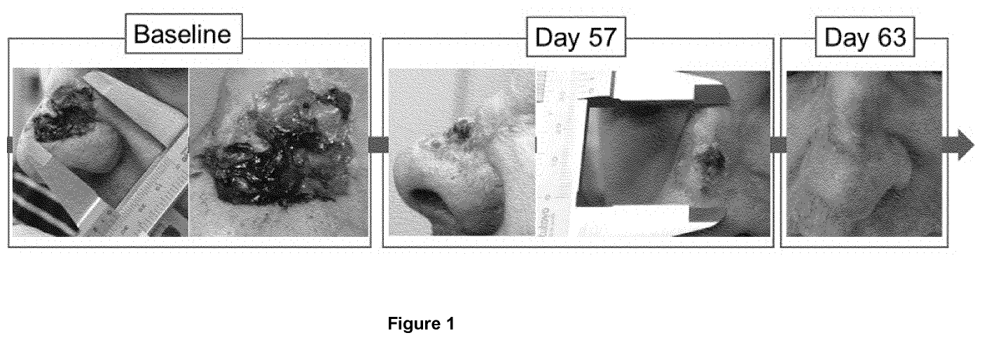

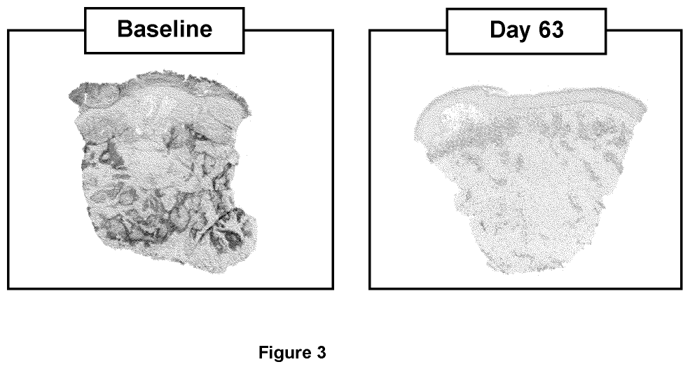

A 55-year-old man presented with an ulcerated and crusted unpigmented tumor measuring 27×25 mm on the left side of the nose causing saddle-nose-like disfiguration (FIG. 1). Histopathology performed on a punch biopsy showed solid and partially (about 30%) sclerodermiform basal cell carcinoma (BCC) revealing a basosquamous component (FIG. 3; left panel). Surgical resection of the tumor would have led to further facial disfiguration to the patient. The BCC tumor was classified as stage IIIB as defined by EADO classification system (Grob et al., 2021).

The patient received four once-weekly intratumoral injections of a mixture of 1.08 mg L19-huIL2 and 200 μg L19-huTNFα, each in a volume of 1 ml. In the second injection, the dose of L19-huIL2 was accidentally increased to 2.17 mg. This is not expected to have had a material impact on the outcome of the treatment and the same outcome is expected for patients treated with a dose of 1.08 mg L19-huIL2 throughout the treatment.

For each injection, the total injection volume was distributed over the tumor mass by performing approximately five to six injections in different parts of the tumor.

### 1.2 Results

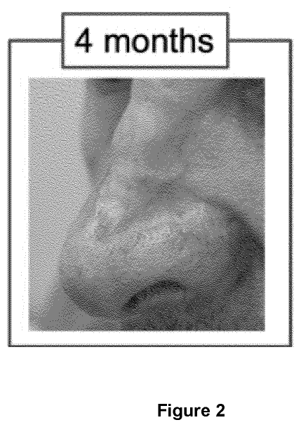

In response to treatment, the tumor decreased in size, progressively converting into a necrotic scab and then to a crust, which eventually fell off. Moreover, a temporary edematous increase of the lesional and perilesional facial skin at the tumor site was also observed. Two months (day 63) after the first injection, the tumor showed a clinical complete response, as well as a pathological complete response to the treatment, and was replaced by a fully epithelialized scar. The fully epithelialized scar was present at a follow-up four months after the first injection, showing no recurrence of the cancer (FIG. 2). The pathological complete response was confirmed by histological analysis of a tissue sample obtained from the site of the original tumor. Specifically, histochemical staining of a punch biopsy taken at day 63 after the first injection showed residual inflammatory lymphohistiocytic, partly granulomatous infiltrates, but an absence of basaloid or sclerodermiform epithelial tumor cells (FIG. 3; right panel). The patient showed no recurrence of the cancer at a follow-up 6 months after the first injection.

The treatment was well-tolerated. The patient experienced mild to moderate episodes of chills and elevated temperature up to 38.5° C. which persisted for up to 36 hours following the injections. To attenuate these cytokine-related symptoms, the patient was given a pre-medication with 1 g metamizole one hour before the injections, starting with the second treatment cycle. Locally, the patient experienced a temporary facial edema persisting for three to four days following the injections.

The clinical and pathological complete response of the basal cell carcinoma (BCC) tumor as a result of the intratumoral injection of the combination of L19-huIL2 and L19-huTNFα was unexpected and demonstrates the surprising efficacy of this combination for the treatment of non-melanoma skin cancers.

Sequence Listing
Amino acid sequence of the L19 CDR1 VH
SEQ ID NO: 1
SFSMS
Amino acid sequence of the L19 CDR2 VH
SEQ ID NO: 2
SISGSSGTTYYADSVKG
Amino acid sequence of the L19 CDR3 VH
SEQ ID NO: 3
PFPYFDY
Amino acid sequence of the L19 CDR1 VL
SEQ ID NO: 4
RASQSVSSSFLA
Amino acid sequence of the L19 CDR2 VL
SEQ ID NO: 5
YASSRAT
Amino acid sequence of the L19 CDR3 VL
SEQ ID NO: 6
QQTGRIPPT
Amino acid sequence of the L19 VH domain
SEQ ID NO: 7
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSS
Amino acid sequence of the linker between VH
and VL
SEQ ID NO: 8
GDGSSGGSGGAS
Amino acid sequence of the L19 VL domain
SEQ ID NO: 9
EIVLTQSPGTLSLSPGERATLSCRASQSVSSSFLAWYQQKPGQAP
RLLIYYASSRATGIPDRFSGSGSGTDFTLTISRLEPEDFAVYYCQ
QTGRIPPTFGQGTKVEIK
Amino acid sequence of the L19 scFv
The linker sequence is underlined.
SEQ ID NO: 10
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGDGSSGGSGGASEIVLTQS
PGTLSLSPGERATLSCRASQSVSSSFLAWYQQKPGQAPRLLIYYA
SSRATGIPDRFSGSGSGTDFTLTISRLEPEDFAVYYCQQTGRIPP
TFGQGTKVEIK
Amino acid sequence of human Interleukin 2
(huIL2)
SEQ ID NO: 11
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT
Amino acid sequence of the linker between
the L19 scFv and IL2
SEQ ID NO: 12
EFSSSSGSSSSGSSSSG
Amino acid sequence of the L19-huIL2 conjugate
The linker sequences are underlined.
SEQ ID NO: 13
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGDGSSGGSGGASEIVLTQS
PGTLSLSPGERATLSCRASQSVSSSFLAWYQQKPGQAPRLLIYYA
SSRATGIPDRFSGSGSGTDFTLTISRLEPEDFAVYYCQQTGRIPP
TFGQGTKVEIKEFSSSSGSSSSGSSSSGAPTSSSTKKTQLQLEHL
LLDLQMILNGINNYKNPKLTRMLTFKFYMPKKATELKHLQCLEEE
LKPLEEVLNLAQSKNFHLRPRDLISNINVIVLELKGSETTFMCEY
ADETATIVEFLNRWITFCQSIISTLT
Amino acid sequence of the soluble form of
the extracellular domain of human TNFα
(huTNFα)
SEQ ID NO: 14
VRSSSRTPSDKPVAHVVANPQAEGQLQWLNRRANALLANGVELRD
NQLVVPSEGLYLIYSQVLFKGQGCPSTHVLLTHTISRIAVSYQTK
VNLLSAIKSPCQRETPEGAEAKPWYEPIYLGGVFQLEKGDRLSAE
INRPDYLDFAESGQVYFGIIAL
Amino acid sequence of the linker between
the L19 scFv and TNFα
SEQ ID NO: 15
EFSSSSGSSSSGSSSSG
Amino acid sequence of the L19-huTNFα conjugate
The linker sequences are underlined.
SEQ ID NO: 16
EVQLLESGGGLVQPGGSLRLSCAASGFTFSSFSMSWVRQAPGKGL
EWVSSISGSSGTTYYADSVKGRFTISRDNSKNTLYLQMNSLRAED
TAVYYCAKPFPYFDYWGQGTLVTVSSGDGSSGGSGGASEIVLTQS
PGTLSLSPGERATLSCRASQSVSSSFLAWYQQKPGQAPRLLIYYA
SSRATGIPDRFSGSGSGTDFTLTISRLEPEDFAVYYCQQTGRIPP
TFGQGTKVEIKEFSSSSGSSSSGSSSSGVRSSSRTPSDKPVAHVV
ANPQAEGQLQWLNRRANALLANGVELRDNQLVVPSEGLYLIYSQV
LFKGQGCPSTHVLLTHTISRIAVSYQTKVNLLSAIKSPCQRETPE
GAEAKPWYEPIYLGGVFQLEKGDRLSAEINRPDYLDFAESGQVYF
GIIAL

### REFERENCES

A number of publications are cited above in order to more fully describe and disclose the invention and the state of the art to which the invention pertains. Full citations for these references are provided below. The entirety of each of these references is incorporated herein.
Balza et al., 2010, Int. J. Cancer, 127, 101
Danielli et al., 2015, Cancer Immunol. Immunother. 64, 113-121
Grob et al., “Position statement on classification of basal cell carcinomas. Part 2: EADO proposal for new operational staging system adapted to basal cell carcinomas”, J Eur Acad Dermatol Venereol, 2021.
Pasche & Neri, 2012, Drug Discov. Today, 17, 583
Pons et al., 2022, “Observational study of a series of basal cell carcinomas: Evaluation of location as a risk factor for recurrence”, J Stomatol Oral Maxillofac Surg 123, 655-659
Schwager et al., 2013, J. Invest. Dermatol. 133, 751-758
Stratigos et al., Eur J Cancer, 2020.
Weide et al., 2011, Cancer—116, 4139-4146
Weide et al., 2014, Cancer Immunol. Immunother. 2, 668-678

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1-22. (canceled)

23. A composition comprising a combination of L19-huTNFα and L19-huIL2 for use in a method of treating a non-melanoma skin cancer in a human patient, wherein the non-melanoma skin cancer is basal cell carcinoma (BCC),
wherein the L19-huTNFα comprises, or consists of, the sequence set forth in SEQ ID NO: 16 and the L19-huIL2 comprises, or consists of, the sequence set forth in SEQ ID NO: 13;
wherein the method comprises injecting the composition intratumorally; and
wherein the non-melanoma skin cancer tumor shows a pathological complete response to the treatment.

24. The composition for use according to claim 23, wherein the BCC is a stage IIA, IIB, IIIA, or IIIB BCC, as defined by the EADO classification system.

25. The composition for use according to claim 24, wherein the BCC is a stage IIIB BCC, defined in accordance with the EADO classification system.

26. The composition for use according to claim 23, wherein the method comprises injecting a dose of the composition comprising:
(i) a maximum amount of 1.08 mg L19-huIL2 and a maximum amount of 200 μg L19-huTNFα; or
(ii) a maximum amount of 2.17 mg L19-huIL2 and a maximum amount of 400 μg L19-huTNFα.

27. The composition for use according to claim 26, wherein the dose comprises:
(i) an amount of 0.27 mg to 1.08 mg L19-huIL2 and an amount of 50 μg to 200 μg L19-huTNFα; or
(ii) an amount of 0.54 mg to 2.17 mg L19-huIL2 and an amount of 100 μg to 400 μg L19-huTNFα.

28. The composition for use according to claim 23, wherein the dose comprises 1.08 mg L19-huIL2 and 200 μg L19-huTNFα.

29. The composition for use according to claim 23, wherein the dose comprises 2.17 mg L19-huIL2 and 400 μg L19-huTNFα.

30. The composition for use according to claim 26, wherein the method comprises injecting a non-melanoma skin cancer tumor located on the nose or in a periorificial region of the head of the patient, the method comprising injecting a dose of the composition comprising:
(a) 25% of the maximum amount of L19-huIL2 and L19-huTNFα as set out in (i) or (ii), where the tumor has a surface area with a longest diameter of >1 cm and ≤2 cm;
(b) 37.5% of the maximum amount of L19-huIL2 and L19-huTNFα as set out in (i) or (ii), where the tumor has a surface area with a longest diameter of >2 cm and ≤3 cm;
(c) 50% of the maximum amount of L19-huIL2 and L19-huTNFα as set out in (i) or (ii), where the tumor has a surface area with a longest diameter of >3 cm and ≤4 cm;
(d) 62.5% of the maximum amount of L19-huIL2 and L19-huTNFα as set out in (i) or (ii), where the tumor has a surface area with a longest diameter of >4 cm and ≤5 cm; or
(e) 75% of the maximum amount of L19-huIL2 and L19-huTNFα as set out in (i) or (ii), where the tumor has a surface area with a longest diameter of >5 cm.

31. The composition for use according to claim 26, wherein the method comprises injecting a non-melanoma skin cancer tumor located on the nose or in a periorificial region of the head of the patient, the method comprising injecting a dose of the composition comprising:
(a) 25% of the maximum amount of L19-huIL2 and L19-huTNFα as set out in (i) or (ii), where the tumor has a surface area with a longest diameter of >1 cm and ≤2 cm;
(b) 37.5% of the maximum amount of L19-huIL2 and L19-huTNFα as set out in (i) or (ii), where the tumor has a surface area with a longest diameter of >2 cm and ≤3 cm;
(c) 50% of the maximum amount of L19-huIL2 and L19-huTNFα as set out in (i) or (ii), where the tumor has a surface area with a longest diameter of >3 cm and ≤5 cm; or
(d) 62.5% of the maximum amount of L19-huIL2 and L19-huTNFα as set out in (i) or (ii), where the tumor has a surface area with a longest diameter of >5 cm.

32. The composition for use according to claim 26, wherein the method comprises injecting a non-melanoma skin cancer tumor located on a part of the body of the patient other than on the nose or in a periorificial region of the head of the patient, the method comprising injecting a dose of the composition comprising:
(a) 37.5% of the maximum amount of L19-huIL2 and L19-huTNFα as set out in (i) or (ii), where the tumor has a surface area with a longest diameter of >1 cm and ≤2 cm;
(b) 50% of the maximum amount of L19-huIL2 and L19-huTNFα as set out in (i) or (ii), where the tumor has a surface area with a longest diameter of >2 cm and ≤3 cm;
(c) 62.5% of the maximum amount of L19-huIL2 and L19-huTNFα as set out in (i) or (ii), where the tumor has a surface area with a longest diameter of >3 cm and ≤4 cm;
(d) 75% of the maximum amount of L19-huIL2 and L19-huTNFα as set out in (i) or (ii), where the tumor has a surface area with a longest diameter of >4 cm and ≤5 cm; or
(e) 100% of the maximum amount of L19-huIL2 and L19-huTNFα as set out in (i) or (ii), where the tumor has a surface area with a longest diameter of >5 cm;
optionally wherein the non-melanoma skin cancer tumor is located on a region of the head of the patient other than on the nose or in a periorificial region, optionally wherein the non-melanoma skin cancer tumor is located on the forehead, cheek, chin, or scalp of the patient.

33. The composition for use according to claim 26, wherein the method comprises injecting a non-melanoma skin cancer tumor located on a part of the body of the patient other than on the nose or in a periorificial region of the head of the patient, the method comprising injecting a dose of the composition comprising:
(a) 37.5% of the maximum amount of L19-huIL2 and L19-huTNFα as set out in (i) or (ii), where the tumor has a surface area with a longest diameter of >1 cm and ≤2 cm;
(b) 50% of the maximum amount of L19-huIL2 and L19-huTNFα as set out in (i) or (ii), where the tumor has a surface area with a longest diameter of >2 cm and ≤3 cm;
(c) 75% of the maximum amount of L19-huIL2 and L19-huTNFα as set out in (i) or (ii), where the tumor has a surface area with a longest diameter of >3 cm and ≤5 cm; or
(d) 100% of the maximum amount of L19-huIL2 and L19-huTNFα as set out in (i) or (ii), where the tumor has a surface area with a longest diameter of >5 cm;
optionally wherein the non-melanoma skin cancer tumor is located on a region of the head of the patient other than on the nose or in a periorificial region, optionally wherein the non-melanoma skin cancer tumor is located on the forehead, cheek, chin, or scalp of the patient.

34. The composition for use according to claim 30, wherein the patient has multiple non-melanoma skin cancer tumors, wherein the method comprises injecting each tumor, in order of decreasing tumor size as measured by the longest diameter of the tumor surface area, with a dose of the composition comprising:
(1a) 25% of the maximum amount of L19-huIL2 and L19-huTNFα as set out in (i) or (ii), where the tumor has a surface area with a longest diameter of >1 cm and ≤2 cm;
(1b) 37.5% of the maximum amount of L19-huIL2 and L19-huTNFα as set out in (i) or (ii), where the tumor has a surface area with a longest diameter of >2 cm and ≤3 cm;
(1c) 50% of the maximum amount of L19-huIL2 and L19-huTNFα as set out in (i) or (ii), where the tumor has a surface area with a longest diameter of >3 cm and ≤4 cm;
(1d) 62.5% of the maximum amount of L19-huIL2 and L19-huTNFα as set out in (i) or (ii), where the tumor has a surface area with a longest diameter of >4 cm and ≤5 cm; or
(1e) 75% of the maximum amount of L19-huIL2 and L19-huTNFα as set out in (i) or (ii), where the tumor has a surface area with a longest diameter of >5 cm;
(2a) 37.5% of the maximum amount of L19-huIL2 and L19-huTNFα as set out in (i) or (ii), where the tumor has a surface area with a longest diameter of >1 cm and ≤2 cm;
(2b) 50% of the maximum amount of L19-huIL2 and L19-huTNFα as set out in (i) or (ii), where the tumor has a surface area with a longest diameter of >2 cm and ≤3 cm;
(2c) 62.5% of the maximum amount of L19-huIL2 and L19-huTNFα as set out in (i) or (ii), where the tumor has a surface area with a longest diameter of >3 cm and ≤4 cm;
(2d) 75% of the maximum amount of L19-huIL2 and L19-huTNFα as set out in (i) or (ii), where the tumor has a surface area with a longest diameter of >4 cm and ≤5 cm; or
(2e) 100% of the maximum amount of L19-huIL2 and L19-huTNFα as set out in (i) or (ii), where the tumor has a surface area with a longest diameter of >5 cm;
(3a) 25% of the maximum amount of L19-huIL2 and L19-huTNFα as set out in (i) or (ii), where the tumor has a surface area with a longest diameter of >1 cm and ≤2 cm;
(3b) 37.5% of the maximum amount of L19-huIL2 and L19-huTNFα as set out in (i) or (ii), where the tumor has a surface area with a longest diameter of >2 cm and ≤3 cm;
(3c) 50% of the maximum amount of L19-huIL2 and L19-huTNFα as set out in (i) or (ii), where the tumor has a surface area with a longest diameter of >3 cm and ≤5 cm; or
(3d) 62.5% of the maximum amount of L19-huIL2 and L19-huTNFα as set out in (i) or (ii), where the tumor has a surface area with a longest diameter of >5 cm; or
(4a) 37.5% of the maximum amount of L19-huIL2 and L19-huTNFα as set out in (i) or (ii), where the tumor has a surface area with a longest diameter of >1 cm and ≤2 cm;
(4b) 50% of the maximum amount of L19-huIL2 and L19-huTNFα as set out in (i) or (ii), where the tumor has a surface area with a longest diameter of >2 cm and ≤3 cm;
(4c) 75% of the maximum amount of L19-huIL2 and L19-huTNFα as set out in (i) or (ii), where the tumor has a surface area with a longest diameter of >3 cm and ≤5 cm; or
(4d) 100% of the maximum amount of L19-huIL2 and L19-huTNFα as set out in (i) or (ii), where the tumor has a surface area with a longest diameter of >5 cm;
until either:
(i) the maximum amount of L19-huIL2 and L19-huTNFα as set out in (i) or (ii) has been injected;
or
(ii) all non-melanoma skin cancer tumors of the patient have been injected.

35. The composition for use according to claim 23, wherein the method comprises injecting more than one dose of the composition into the non-melanoma skin cancer tumor, and wherein the doses are administered to the patient once weekly.

36. The composition for use according to claim 35, wherein the method comprises injecting an initial dose of the composition and one or more further doses of the composition intratumorally, and wherein one or more of the further doses comprises more or less L19-huIL2 and/or L19-huTNFα than the initial dose.

37. The composition for use according to claim 36, wherein one or more of the further doses comprises double or half the amount of L19-huIL2 and/or L19-huTNFα relative to the initial dose, wherein the one or more further doses administered do not exceed the maximum amount of L19-IL2 and L19-TNFα set out below:
(i) a maximum amount of 1.08 mg L19-huIL2 and a maximum amount of 200 μg L19-huTNFα; or
(ii) a maximum amount of 2.17 mg L19-huIL2 and a maximum amount of 400 μg L19-huTNFα.

38. The composition for use according to claim 23, wherein the method is a neoadjuvant method.


SEQUENCE LISTING (16 sequences)

SEQ ID NO: 1 - protein, 5 aa, synthetic construct
SFSMS

SEQ ID NO: 2 - protein, 17 aa, synthetic construct
SISGSSGTTYYADSVKG

SEQ ID NO: 3 - protein, 7 aa, synthetic construct
PFPYFDY

SEQ ID NO: 4 - protein, 12 aa, synthetic construct
RASQSVSSSFLA

SEQ ID NO: 5 - protein, 7 aa, synthetic construct
YASSRAT

SEQ ID NO: 6 - protein, 9 aa, synthetic construct
QQTGRIPPT

SEQ ID NO: 7 - protein, 116 aa, synthetic construct [sequence omitted]

SEQ ID NO: 8 - protein, 12 aa, synthetic construct
GDGSSGGSGGAS

SEQ ID NO: 9 - protein, 108 aa, synthetic construct [sequence omitted]

SEQ ID NO: 10 - protein, 236 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 11 - protein, 133 aa, synthetic construct [sequence omitted]

SEQ ID NO: 12 - protein, 17 aa, synthetic construct
EFSSSSGSSSSGSSSSG

SEQ ID NO: 13 - protein, 386 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 157 aa, synthetic construct [sequence omitted]

SEQ ID NO: 15 - protein, 17 aa, synthetic construct
EFSSSSGSSSSGSSSSG

SEQ ID NO: 16 - protein, 410 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL